(19) United States
(12) Patent Application Publication
(10) Pub. No.: US 2009/0017513 A1
(43) Pub. Date: Jan. 15, 2009

(54) PROCESS FOR PRODUCING HYDROCARBON MOLECULES FROM RENEWABLE BIOMASS

(75) Inventors: James C. Bell, Tifton, GA (US); James J. Lever, IV, Tifton, GA (US); Deborah D. Layfield, Tifton, GA (US); Kurt E. Seigler, Tifton, GA (US)

Correspondence Address: HUNTON & WILLIAMS/NEW YORK;INTELLECTUAL PROPERTY DEPT., 1900 K STREET, N.W., SUITE 1200, WASHINGTON, DC, 20006-1109, US

(73) Assignee: Georgia Belle Plantation, Inc., Tifton, GA (US)

(21) Appl. No.: 12/147,880

(22) Filed: Jun. 27, 2008

Related U.S. Application Data
(60) Provisional application No. 60/949,651, filed on Jul. 13, 2007.

(51) Int. Cl. C12P 5/00

(52) U.S. Cl. 435/166

(57) ABSTRACT

Provided is a process for producing hydrocarbon molecules from biomass utilizing microorganisms that are resistant to extreme heat and pressure, that comprise nucleic acid molecules encoding enzymes which degrade compounds selected from the group consisting of cellulose, lignin, xylan and hemicellulose, and which are capable of generating hydrocarbon molecules from the degraded cellulose, lignin, xylan and hemicellulose.

### PRIORITY DATA

This application claims priority under 35 U.S.C. §119 to Provisional Patent Application No. 60/949,651, filed on Jul. 13, 2007.

### FIELD OF INVENTION

This invention relates to methods of using microorganisms that produce enzymes to breakdown cellulose, hemicellulose, xylan and/or lignin to also synthesize hydrocarbon molecules.

Biomass is defined as the total of all plant growth on earth. Biomass can also be described as the accumulation and storage of the sun's energy by plants; it is estimated that 140 billion metric tons of biomass are synthesized by photosynthesis using light energy from sunlight, carbon dioxide and water. By definition, organic materials produced by plants, include leaves, roots, seeds, stalks, as well as materials derived from plants, such as animal manure are all biomass. A small portion of this biomass is consumed as food, e.g., as starch, sugar, oil, used as lumber or manufactured into consumer products. Generally, the term biomass refers to organic materials that are not used as food or for consumer products.

The agricultural and forestry industries produce billions of tons of biomass annually as reported by the United States Department of Agricultural (USDA) study commonly referred to as the ‘Billion Ton Study.’ The largest part of all the biomass produced by the agricultural and forestry industries is considered waste and it is estimated by the USDA that over 1.3 billion tons of this waste is easily available annually. This amount of biomass is enough to produce biofuels to meet more than one-third of the current demand for transportation fuels in the U.S. Forest biomass waste is comprised of the limbs, leaves and tops of trees and agricultural biomass waste is comprised of the stalks of plants, such as corn stalks, straw, seed hulls, sugarcane leavings, bagasse, nutshells, and every other unused portion of the plants grown, as well as manure from cattle, poultry and hogs.

Agricultural and forestry waste is of primary consideration because of its quantity and availability, but biomass waste can come from a variety of other sources. Other sources biomass organic raw material include wood material, e.g., wood, bark, sawdust, timber slash and mill scrap, residue from wood processing mills and pulp and mill waste, wood from construction and demolition sites; municipal waste streams, such as waste paper and yard clippings; and energy crops, e.g., poplars, willows, switchgrass, alfalfa, prairie bluestem, corn starch and soybean oil. Construction and demolition produce many millions of tons of wood material waste annually. Construction wood waste is defined as any unusable wood remaining after project completion. Demolition wood waste is defined as all wood products removed from a building or site during the demolition process. Inert landfills are expensive and rapidly filling to capacity with wood materials removed during site preparation, landscaping and general lawn maintenance. Utilizing these sources of biomass will greatly reduce the waste stream into inert landfills, diminish green house gas emissions (methane and carbon dioxide) from disposal systems and thus lower the cost of operation for government and private enterprise.

Forest and agricultural biomass is a valuable natural source of renewable organic matter, and hence a renewable source of fuel and energy because is sustainably available annually. The use of biomass to provide renewable energy, such as biomass-derived fuels, is a way to reduce the need and dependence on foreign oil and gas imports.

Woody and non-woody plants, such as grass, are composed structurally of lignocellulose, which consists of lignin and carbohydrates, which are mostly cellulose and hemicellulose fibers. Forest and agricultural biomass is thus a lignocellulosic material. Solid biomass may be converted to biomass fuels by fermentation or be chemically liquefied by pyrolysis, hydrothermal liquefaction, or other thermochemical technologies. Gasification, another way in which biomass is may be converted to biomass fuels, involves heating the biomass with little or no oxygen to gasify it to a mixture of carbon monoxide and hydrogen; such gas is referred to as synthesis gas or ‘syngas.’ Methane gas is produced by anaerobic microbial digestion of human and animal waste for local energy use in China. Methane accumulation in manure storage areas poses certain hazards: as an odorless gas, it may be difficult to detect but if its accumulates in high concentrations at the top of manure pits it may cause asphyxiation, and because it is flammable, it poses a risk of explosion.

Current chemical processing techniques can not efficiently convert all components of biomass into liquid fuels that can be directly incorporated into our fuel production system for subsequent use in existing fleets of automobiles, trucks, trains and aircraft. It would be desirable to provide new approaches to directly convert cellulose and lignin into hydrocarbons that can be refined into gasoline, diesel fuel and aviation fuel suitable for use in current engine systems.

The Energy Policy Act of 2005 increase to 7.5 billion gallons the amount of biofuels to be used annually by 2012. Biofuel, e.g., in liquid or gas form, is fuel derived from biomass.

E10, sometimes called gasohol, is a mixture of 10% ethanol and 90% gasoline, for which the ethanol, also called bio-ethanol, is often made by fermenting agricultural crops, e.g., corn, or crop wastes; however this process is expensive. An alternative gasohol is a mixture of 97% gasoline and 3% methanol (wood alcohol); but production of methanol also is expensive, the alcohol is toxic and corrosive, and its emissions produce formaldehyde, a carcinogen.

Currently, ethanol produced by fermentation is produced, e.g., from starch biomass, by enzymatic hydrolysis into glucose followed by fermentation of the glucose by yeast into ethanol. It would be desirable to provide hydrocarbon molecules for the production of fuel from the abundantly and renewably available biomass waste. It further would be appealing to provide a method of producing hydrocarbon molecules from biomass that avoids a processing step used in fermentation (hydrolysis into glucose) and more importantly, prevents a loss of about 40% carbon from the biomass.

Throughout this description, including the foregoing description of related art, any and all publicly available documents described herein, including any and all U.S. patents, are specifically incorporated by reference herein in their entirety. The foregoing description of related art is not intended in any way as an admission that any of the documents described therein, including pending United States patent applications, are prior art to the present invention. Moreover, the description herein of any disadvantages associated with the described products, methods, and/or apparatus, is not intended to limit the invention. Indeed, aspects of the invention may include certain features of the described products, methods, and/or apparatus without suffering from their described disadvantages.

### SUMMARY

In an embodiment, a process for producing hydrocarbon molecules from biomass is provided, wherein said process comprises: pre-processing the biomass by physical or mechanical breakdown, chemical degradation or a combination of physical or mechanical breakdown and chemical degradation to form a biomass fiber slurry; introducing the biomass fiber slurry into a first stage bioreactor; introducing microorganisms to the biomass fiber slurry in the first stage bioreactor, said microorganisms (a) being resistant to extreme heat and pressure, (b) comprising nucleic acid molecules encoding enzymes which degrade compounds selected from the group consisting of cellulose, lignin, xylan and hemicellulose, and (c) capable of generating hydrocarbon molecules from degraded cellulose, lignin, xylan and hemicellulose; incubating the microorganisms with the biomass fiber slurry in the first stage bioreactor at a temperature of between about 70° F. and about 120° F., an atmospheric pressure of near-vacuum, i.e., between about 0.1 atm or about 0.2 atm and about 3 atm, and a pH (depending on the type of microorganisms used) of between about 0.5 and about 2.0 for microorganisms which are acidophilic, i.e., grow well in an acid medium, and between about 7.0 and about 9.0 for microorganisms which are basophilic, i.e., thrive in a basic culture environment, for about 180 days to allow (a) the microorganisms to produce the enzymes which degrade compounds selected from the group consisting of cellulose, lignin, xylan and hemicellulose, (b) the produced enzymes to degrade the cellulose, lignin, xylan and hemicellulose and (c) the microorganisms to generate hydrocarbon molecules from the degraded cellulose, lignin, xylan and hemicellulose; removing a hydrocarbon slurry from the first stage bioreactor, said hydrocarbon slurry comprising hydrocarbon molecules; an aqueous solution comprising enzymes produced by the microorganisms; and sludge comprising microorganisms and non-hydrocarbon byproducts of enzymatic degradation of cellulose, lignin, xylan and hemicellulose; and separating the hydrocarbon molecules from the hydrocarbon slurry.

In another embodiment, the cellulose degrading microorganisms are genetically engineered for optimal bioprocessing condition tolerance, i.e., high temperature of between about 70° F. and about 120° F., pressure near-vacuum and, depending upon the type of microorganisms used, a low pH (for acidophilic microorganisms) or high pH (for basophilic pH), little oxygen or no oxygen (anaerobic conditions) and for the production of hydrocarbon.

These and other embodiments will become readily apparent to those skilled in the art upon review of the detailed description that follows.

### BRIEF DESCRIPTION OF DRAWINGS

FIGS. 1A-1B. FIG. 1A provides a flow chart of the provided bioprocessing system used to produce hydrocarbon from agricultural and forest residues, i.e., biomass. FIG. 1B depicts a flow chart of research activities designed to make the process of hydrocarbon production from biomass more efficient, wherein microorganisms which produce cellulose degrading enzymes will be used to produce genetic libraries for identification of microbial enzymes and microorganism strains for optimal bio-processing tolerance and hydrocarbon production and to genetically engineer the optimal microbes candidates for use in hydrocarbon production process.

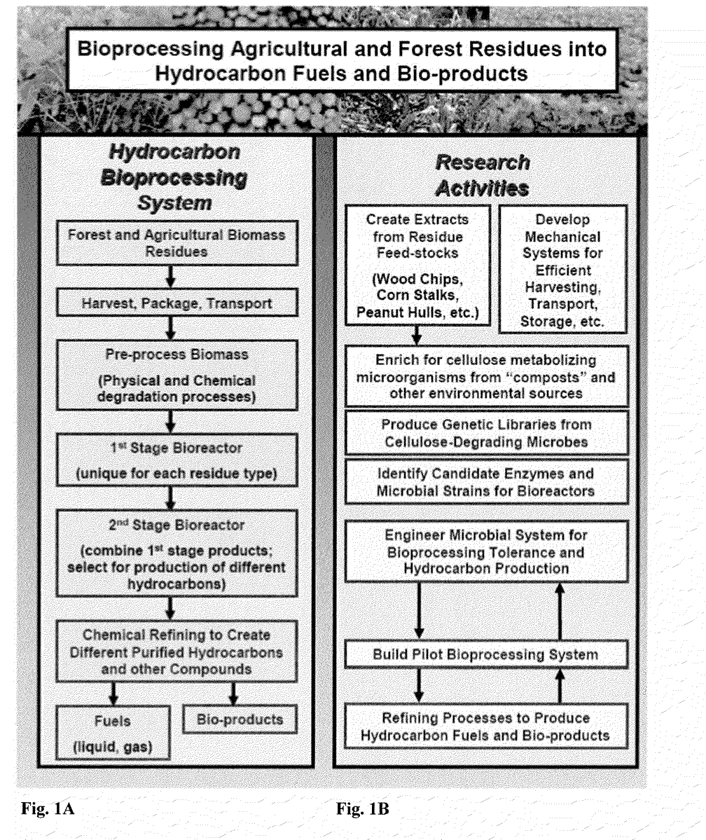

FIG. 2 illustrates (a) the bioprocessing of steps of the provided process used to produce hydrocarbon molecules and bioproducts (and cellulytic enzymes, among others) from biomass obtained from agricultural and forest residue in sequence and (b) the reusability of the sludge comprising microbe communities and byproducts in this process.

### DETAILED DESCRIPTION OF THE INVENTION

A process for converting agricultural and forestry waste biomass into hydrocarbon molecules, commonly referred to as crude oil, is provided herein. This process utilizes microorganisms to chemically or enzymatically transform cellulose, lignin, and hemicellulose from plant material into hydrocarbon molecules. In an embodiment genetically-modified bacteria will be utilized in the process. Bioengineered hydrocarbons may be a path to cost-efficient, renewable and environmentally responsible hydrocarbons which are the raw materials necessary for many other manufactured products, such as fuels, polymers, chemicals and solvents.

The primary product of the provided process, hydrocarbons, can be used as fuels, polymers, chemicals and solvents. Other products of the reaction, such as oxygen, pure distilled water, and carbon dioxide, can also be marketed.

In an embodiment, a process for producing hydrocarbon molecules from biomass is provided, wherein said process comprises: pre-processing the biomass by physical or mechanical breakdown, chemical degradation or a combination of physical or mechanical breakdown and chemical degradation to form a biomass fiber slurry; introducing the biomass fiber slurry into a first stage bioreactor; introducing microorganisms to the biomass fiber slurry in the first stage bioreactor, said microorganisms (a) being resistant to extreme heat and pressure, (b) comprising nucleic acid molecules encoding enzymes which degrade compounds selected from the group consisting of cellulose, lignin, xylan and hemicellulose, and (c) capable of generating hydrocarbon molecules from degraded cellulose, lignin, xylan and hemicellulose; incubating the microorganisms with the biomass fiber slurry in the first stage bioreactor at a temperature of between about 70° F. and about 120° F., an atmospheric pressure of near-vacuum, i.e., between about 0.1 atm or about 0.2 atm and about 3 atm, and a pH (depending on the type of microorganisms used) of between about 0.5 and about 2.0 for microorganisms which are acidophilic, i.e., grow well in an acid medium, and between about 7.0 and about 9.0 for microorganisms which are basophilic, i.e., thrive in a basic culture environment, for about 180 days to allow (a) the microorganisms to produce the enzymes which degrade compounds selected from the group consisting of cellulose, lignin, xylan and hemicellulose, (b) the produced enzymes to degrade the cellulose, lignin, xylan and hemicellulose and (c) the microorganisms to generate hydrocarbon molecules from the degraded cellulose, lignin, xylan and hemicellulose; removing a hydrocarbon slurry from the first stage bioreactor, said hydrocarbon slurry comprising hydrocarbon molecules; an aqueous solution comprising enzymes produced by the microorganisms; and sludge comprising microorganisms and non-hydrocarbon byproducts of enzymatic degradation of cellulose, lignin, xylan and hemicellulose; and separating the hydrocarbon molecules from the hydrocarbon slurry.

In an embodiment of the provided process, the biomass is sterilized before being introduced into the first stage bioreactor. In another embodiment the process further comprises filtering the hydrocarbon molecules to remove foreign or unwanted substances. In an embodiment the process further comprises refining the filtered hydrocarbon molecules to produce liquid hydrocarbon fuel.

In an embodiment, the process further comprising removing from the hydrocarbon slurry (a) the aqueous solution comprising enzymes produced by the microorganisms and (b) the sludge comprising microorganisms and non-hydrocarbon byproducts of enzymatic degradation of cellulose, lignin, xylan and hemicellulose. In another embodiment, the process further comprises introducing the removed sludge comprising microorganisms into a second stage bioreactor to cultivate the microorganisms. In a further embodiment, the process further comprises removing the cultivated microorganisms from the second stage bioreactor and re-introducing the cultivated microorganisms into the first stage bioreactor. In yet another embodiment, the process further comprises removing any remaining hydrocarbon from the aqueous solution comprising enzymes produced by the microorganisms. In an embodiment, the process further comprises removing from the first stage bioreactor byproducts selected from the group consisting of oxygen, water, e.g., distilled water may be produced under low pressure, and carbon dioxide.

In another embodiment, the process comprises microorganisms, which may be selected from the group consisting of mixed ruminal microorganisms, a cellulolytic microorganism, e.g., a cellulotyic bacterial species, a hemicellulose-degrading bacterial species from an insect gut, a cellulose-degrading fungus from the gut of an insect, a cellulotyic fungus, a hemicellulotyic fungus, a xylan-degrading microorganism, a bacteria from the gut of a wood eating insect, a bacteria from the gut of Loricardiid catfish Panaque, a fungus from the gut of Loricardiid catfish Panaque, a lignan-degrading bacteria isolated from the soil, but is not limited thereto. (See, e.g., http://www.wzn.tum.de/mbiotec/cellmo.htm and references cited therein, including a server containing all known cellulase sequences at URL: http://amb.cnrs-mrs.fr/cazy/CAZY/index.html; Howard et al. African J of Biotech. Vol. 2, No. 12, December, 2003 pp. 602-619; S. B. Leschine, “Cellulase Degradation in Anaerobic Environments,” Annu. Rev. Microbiol. 1995, 49:399-426; WO 2006/003009 A2, entitled New Esterases from Rumen, published 12 Jan. 2006; J. A. Nelson, et al. (1999) “Wood-eating catfishes of the genus Panaque: gut microflora and cellulolytic enzyme activities,” Journal of Fish Biology 54 (5), 1069-1082; M. P. Bryant, “Bacterial Species of the Rumen,” Microbiol. Mol. Biol. Rev. 23 (3): 125-153, 1959; J. Palmerston et al., “The Effects of Adding Rumen Fluid To the Anaerobic Digestion of Jose Tall Wheatgrass,” Aug. 3, 2006 at http://ysp.ucdavis.edu/Research06/PalmerstonJ/default.html, W. Jones et al., “Methanogens and the Diversity of Archaebacteria,” Microbol. Rev. Vol. 51, No. 1, p. 135-177 (1987); all of which are hereby incorporated by reference in their entirety into the present specification).

In an embodiment of the process, a ruminal microorganism from the mixed ruminal microorganisms may be an anaerobic fungi, an anaerobic protozoa, an anaerobic bacteria or an anaerobic Archaebacteria (Archaea). In a further embodiment, the anaerobic Archaebacteria (Archaea) may be a strain of Methanobrevibacter ruminatum. Other methanogens may be used in further embodiments, See e.g., W. Jones et al., “Methanogens and the Diversity of Archaebacteria,” Microbol. Rev. Vol. 51, No. 1, p. 135-177 (1987); all of which is hereby incorporated by reference in its entirety into the present specification.

In another embodiment, a ruminal microorganism from the mixed ruminal microorganisms may be Alicyclobacillus acidocaldarius, Eubacteria, Ruminococcus albus, R. flavefaciens, Butyrivibrio fibrisolvens, Fibrobacter succinogenes and Selenomonas ruminantium, but is not limited thereto, e.g., See M. P. Bryant, “Bacterial Species of the Rumen,” Microbiol. Mol Biol. Rev. 23 (3): 125-153, 1959; S. B. Leschine, “Cellulase Degradation in Anaerobic Environments,” Annu. Rev. Microbiol. 1995, 49:399-426; WO 2006/003009 A2, entitled New Esterases from Rumen, published 12 Jan. 2006, all of which are hereby incorporated by reference in their entirety into the present specification.

In a further embodiment, the biomass used may be agricultural waste or forestry waste. In another embodiment, the biomass may be waste wood from a construction site or a demolition site. In yet another embodiment, the biomass is municipal waste. In an embodiment, the biomass may be pulp mill waste or pulp wood waste. In a still further embodiment, the biomass may be farming debris or yard waste.

In an embodiment the microorganisms may be genetically engineered. In another embodiment, strain of genetically engineered microorganism produces one length of hydrocarbon molecule. In another embodiment, the genetically engineered microorganisms may be selected from the group consisting of mixed ruminal microorganisms, a cellulolytic microorganism, e.g., a cellulotyic bacterial species, a hemicellulose-degrading bacterial species from an insect gut, a cellulose-degrading fungus from the gut of an insect, a cellulotyic fungus, a hemicellulotyic fungus, a xylan-degrading microorganism, a bacteria from the gut of a wood eating insect, a bacteria from the gut of Loricardiid catfish Panaque, a fungus from the gut of Loricardiid catfish Panaque, a lignan-degrading bacteria isolated from the soil, but not limited thereto, See, e.g., the publications incorporated herein by reference in their entirety supra.

In a further embodiment, a ruminal microorganism from the mixed ruminal microorganisms may be an anaerobic fungi, an anaerobic protozoa, an anaerobic bacteria or an anaerobic Archaebacteria (Archaea). In an embodiment, the anaerobic Archaebacteria (Archaea) may be a strain of Methanobrevibacter ruminatum.

In another embodiment, the ruminal microorganism from the mixed ruminal microorganisms may be Alicyclobacillus acidocaldarius, Ruminococcus albus, R. flavefaciens, Butyrivibrio fibrisolvens, Fibrobacter succinogenes or Selenomonas ruminantium.

In an embodiment, the cellulolytic microorganism may be a strain of Eubacteria, a strain of Clostridium, a strain of Ruminococcus, a strain of Caldocellulosiruptor, a strain of Bacteroides, a strain of Acetivibrio, a strain of Thermoactinomyces, a strain of Caldibacillus, a strain of Bacillus, a strain of Acidothermus, a strain of Cellulomonas, a strain of Curtobacterium, a strain of Micromonospora, a strain of Actinoplanes, a strain of Streptomyces, a strain of Thermobifida, a strain of Thermonospora, a strain of Microbispora, a strain of the family Streptosporangiaceae, a strain of Fibrobacter, a strain of Sporocytophaga, a strain of Cytophaga, a strain of Flavobacterium, a strain of Achromobacter, a strain of Xanthomonas, a strain of Cellvibrio, a strain of Pseudomonas or a strain of Myxobacter.

In another embodiment, the hydrocarbon molecule produced by the provided method may comprise from one to twenty-two carbon atoms. Hydrocarbons with a longer chain length, i.e., over twenty-two carbon atoms, may also be produced. In an embodiment, the hydrocarbon molecule produced may comprise from four to twelve carbon atoms. In a further embodiment, the hydrocarbon molecule may comprise eight carbon atoms, e.g., may be octane.

In yet another embodiment, the process comprises manufacturing a polymer, a plastic, a chemical or a solvent from the hydrocarbon molecules produced by the microorganisms which breakdown cellulose, hemicellulose, lignin and xylan. In an embodiment, the cellulose/hemicellulose/lignan/xylan-degrading microorganisms may be genetically engineered to tolerate extreme conditions of bioreactors, such as high temperature of between about 70° F. and about 120° F., low atmospheric pressure, i.e., at near-vacuum, e.g., from about 0.1 atm or about 0.2 atm to about 3 atm, little or no (anaerobic) oxygen, and either a low pH of about 0.5 to about 2.0 for acidophilic microorganisms or a pH of about 7.0 to about 9.0 for basophilic microorganisms. In a further embodiment the microorganisms are genetically engineered to produce hydrocarbons, e.g., via pathways that do not include breaking the cellulosic compounds down to small carbon chain sugars, such as occurs in fermentation of ethanol. However, microorganisms which breakdown cellulose to C4 or C5 sugars may be used in the process provided herein if such a step enhances the food for the hydrocarbon producing microorganisms. For example, a pathway for hydrocarbon production may include a synthesis route as is used by methanogens in ruminants.

Alternatively, products of cellulose/hemicellulose/lignan/xylan-degrading microorganisms may undergo hydrocarbon biosynthetic pathways whose mechanisms are unknown, but vary in different microorganisms, or are synthesized by pathways including, but not limited to, reduction of organic compounds derived from decarboxylation, elongation-decarboxylation, or decarboxylation-condensation reactions of fatty acids, as described by T. G. Tornabene, “Microorganisms as hydrocarbon producers,” in New Trends in research and utilization of solar energy through biological systems, Birkhäuser Verlag Basel pp. 49-52 (1982), which is hereby incorporated by reference in its entirety into the present specification.

### Biomass Handling

The biomass raw material is collected at the source of its production, e.g., on farmland or at a pulp mill, and mechanically reduced from its original state into a pulverized condition that exposes as much of the substrate as possible to bacterial and enzymatic contact. In this form, the biomass can be easily transported either by truck, train or pipeline to the bioreactor facility. The biomass upon arrival at the bioreactor facility has water added to reach optimum handling consistency. Subsequently, the biomass slurry is pumped into bioreactors for further processing.

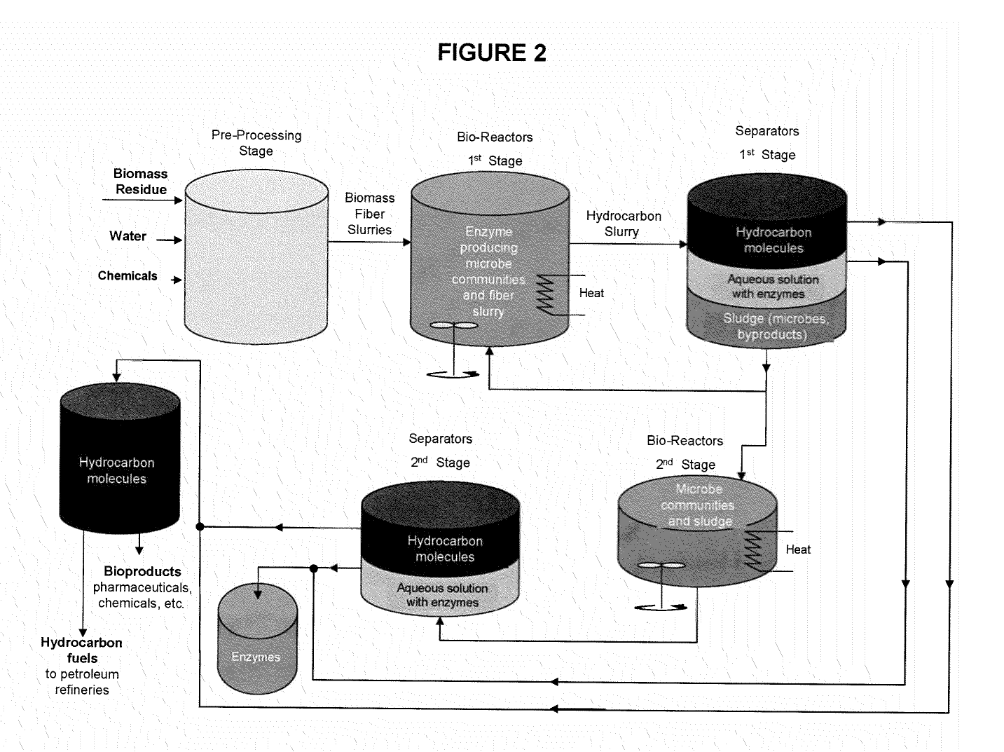

Depending upon the origin, the biomass may need to be pre-processed to achieve necessary size, moisture content, and consistency. The biomass material is sterilized to remove unwanted bacteria from the slurry and then is placed into a bioreactor to begin the provided process; a multi-stage sterilization, called flash heating, is used for sterilization. Genetically-engineered bacteria will be added to the biomass material in the bioreactor. Enzymes produced by the bacteria breakdown cellulose, lignin, and hemicellulose present in the biomass into useable elements that are transformed through bacterial action into hydrocarbons and other products. These products are captured and removed from the bioreactor as illustrated in FIG. 2.

Initially, cellulose/hemicellulose/lignin/xylan-degrading microorganisms that are resistant to extreme heat and pressure will be identified and isolated. Microorganisms which degrade cellulose, hemicellulose, lignan and xylan are known to one of skill (See, e.g. http://www.wzn.tum.de/mbiotec/cellmo.htm and references cited therein, including a server containing all known cellulase sequences at URL: http://amb.cnrs-mrs.fr/cazy/CAZY/index.html; Howard et al. African J. of Biotech. Vol. 2, No. 12, December, 2003 pp. 602-619; S. B. Leschine, “Cellulase Degradation in Anaerobic Environments,” Annu. Rev. Microbiol. 1995, 49:399-426; WO 2006/003009 A2, entitled New Esterases from Rumen, published 12 Jan. 2006; J. A. Nelson, et al. (1999) “Wood-eating catfishes of the genus Panaque: gut microflora and cellulolytic enzyme activities,” Journal of Fish Biology 54 (5), 1069-1082; M. P. Bryant, “Bacterial Species of the Rumen,” Microbiol. Mol. Biol. Rev. 23 (3): 125-153, 1959; J. Palmerston et al., “The Effects of Adding Rumen Fluid To the Anaerobic Digestion of Jose Tall Wheatgrass,” Aug. 3, 2006 at http://ysp.ucdavis.edu/Research06/PalmerstonJ/default.html, W. Jones et al., “Methanogens and the Diversity of Archaebacteria,” Microbol. Rev. Vol. 51, No. 1, p. 135-177 (1987); all of which are hereby incorporated by reference in their entirety into the present specification).

A genetic library of the microorganisms will be constructed and the library screened for enzymes that convert cellulose and other plant structural components into organic compounds. See, e.g., Sections: Isolation of Fibrolytic Enzymes, Preparation of Anaerobic Bacterial Plasmids Suitable for Gene Insertion, Location of Gene Control Factors Located Externally to the Enzyme-Coding Sequences, Integration of Introduced Genes into the Chromosome of the Host Bacterium, Direct Transformation of Rumen Anaerobes, and The Requirements for Research once Recombinant Rumen Bacteria are Developed in “Methods of Modifying Rumen Bacteria” at Appendix A of Application of biotechnology to nutrition of animals in developing countries, FAO Animal Production Health Papers-90, 1991, found on-line at http://www.foa.org/DOCREP/004/T0423E/T0423E10.htm, which is hereby incorporated by reference in its entirety into the present specification.

The complex rumen (e.g., in cows, sheep and other ruminants) microbiome is a unique genetic resource for microbial plant cell wall-degrading enzymes, which may be used for genetically engineering microorganisms for the process provided herein, because the rumen/gastrointestinal tract harbors an estimated 500-1000 native microbial species, of which less than 10% have been cultivated and characterized. B. A. White, et al., “The Rumen Biome, A View through the Fistula,” Speaker Presentation, Second Annual DOE Joint Genome Institute User Meeting, Mar. 28-30, 2007, Walnut Creek, Calif., which is hereby incorporated by reference in its entirety into the present specification.

### Biomass Reduction and Transformation Through Enzymatic and Bacterial Action Into Hydrocarbon Molecules

After the bioreactor is sufficiently filled, genetically modified bacteria and enzymes will be introduced into the biomass slurry. The bacteria and enzymes begin to break down the cellulose, lignin and hemicellulose into their most basic molecules. As the enzymes reduce the cellulose, lignin and hemicellulose, the bacteria use these released compounds for energy, cellular growth and reproduction. The waste products produced by the bacteria are the hydrocarbon molecules, the desired products of the process described herein. Each genetically modified strain of bacteria produces one length of hydrocarbon molecule. This will enable the production of specific length hydrocarbon molecules for further processing into higher value products. These products can be polymers, plastics, fuels, chemicals and solvents.

### Capture and Storage of Hydrocarbon Products

All the hydrocarbon molecules that are produced through the bacterial and enzymatic action are lighter than the water or biomass residue remaining in the bioreactor. As shown in FIG. 2, the hydrocarbon molecules are skimmed, extracted from the bioreactor, and collected in a storage facility. The final process is to filter the hydrocarbon to remove any foreign or unwanted substances.

### By-Products of the Process and Uses of the By-Products

In addition to the produced hydrocarbon molecules of various chain length, useful byproducts of the provided process include oxygen, clean water, e.g., distilled water produced under low pressure, carbon dioxide and trace elements, all of which may be collected for use in other industries.

While the amount from the production of hydrocarbon may be very limited, the by-products have a use and value of their own and are not considered waste. These products are either captured and resold or recycled and reused in the case of the water, e.g., distilled water, used to produce the bio-mass slurry. The by-products of the process are oxygen, carbon dioxide and water, which may be produced as distilled water under low pressure bioreactor conditions.

### Uses of Hydrocarbon Products

The end products that are manufactured from hydrocarbon molecules are extensive and touch every part of business, as well as an individual's personal and professional life. Since each strain of bacteria produces only one length of hydrocarbon molecule, only those bacteria that produce the hydrocarbon molecules with the highest demand will be utilized. The high-demand hydrocarbon molecules may include the hydrocarbon molecules that are the basis for polymers, plastics, fuels, chemicals and solvents.

The cellulose-degrading process provided utilizes renewable waste materials available throughout the United States to yield a product in high demand-hydrocarbon molecules. Production of hydrocarbon from this renewable natural resource has both environmental and economic advantages. This environmentally-responsible production process utilizes agricultural and forestry biomass waste, reduces both municipal waste streams, and the release of greenhouse gases into the atmosphere. Since the process utilizes sustainably available and readily-available waste as raw materials, the bioengineered hydrocarbon may be a cost-efficient alternative to foreign crude, increasing the United States' self-sufficiency.

Additional benefits of the provided process include the use of a bioreactor, which is a closed system, that will eliminate the escape of greenhouse gases such as methane and carbon dioxide into the atmosphere. Other advantages include the reduction of landfill usage and landfills themselves, decreasing of the municipal waste stream, with a concomitant lower cost of biomass waste disposal, thereby improving the community economically. Further gain from the provided process is the sequestration of carbon, i.e., more carbon energy will be available compared to ethanol production by fermentation. Rural economies also will benefit from thriving agricultural and forest industries as a result of the production, harvesting, packaging and transporting of renewable biomass, which will spur new and/or additional employment associated with the bioprocessing system. Additional forests will have to be planted; handling stations for pulp mill, farming debris, household yard waste and other sources of biomass, will have to be created; pumping stations will have to be constructed to deliver pulp mill waste to the bioreactors; railroad transportation of biomass to bioreactor facilities will increase, which may result in additional railroads being built; and bioreactors will be constructed and operated, all of which will spur economic development. Ultimately, a domestic production of hydrocarbon will reduce U.S. dependence on foreign oil and gas and lead to enhanced national security.

Although the invention has been described with reference to various embodiments and examples, those skilled in the art recognize that various modifications may be made to the invention without departing from the spirit and scope thereof.

We claim:

1. A process for producing hydrocarbon molecules from biomass, said process comprising:
pre-processing the biomass by physical or mechanical breakdown, chemical degradation or a combination of physical or mechanical breakdown and chemical degradation to form a biomass fiber slurry;
introducing the biomass fiber slurry into a first stage bioreactor;
introducing microorganisms to the biomass fiber slurry in the first stage bioreactor, said microorganisms (a) being resistant to extreme heat and pressure, (b) comprising nucleic acid molecules encoding enzymes which degrade compounds selected from the group consisting of cellulose, lignin, xylan and hemicellulose, and (c) capable of generating hydrocarbon molecules from degraded cellulose, lignin, xylan and hemicellulose;
incubating the microorganisms with the biomass fiber slurry in the first stage bioreactor at a temperature of between about 70° F. and about 120° F., an atmospheric pressure of near-vacuum, and a pH of between about 0.5 and about 2.0 for microorganisms which are acidophilic or a pH of between about 7.0 and about 9.0 for microorganisms which are basophilic for about 180 days to allow (a) the microorganisms to produce the enzymes which degrade compounds selected from the group consisting of cellulose, lignin, xylan and hemicellulose, (b) the produced enzymes to degrade the cellulose, lignin, xylan and hemicellulose and (c) the microorganisms to generate hydrocarbon molecules from the degraded cellulose, lignin, xylan and hemicellulose;
removing a hydrocarbon slurry from the first stage bioreactor, said hydrocarbon slurry comprising hydrocarbon molecules; an aqueous solution comprising enzymes produced by the microorganisms; and sludge comprising microorganisms and non-hydrocarbon byproducts of enzymatic degradation of cellulose, lignin, xylan and hemicellulose; and
separating the hydrocarbon molecules from the hydrocarbon slurry.

2. The process of claim 1, wherein the biomass is sterilized before being introduced into the first stage bioreactor.

3. The process of claim 1, further comprising filtering the hydrocarbon molecules to remove foreign or unwanted substances.

4. The process of claim 4, further comprising refining the filtered hydrocarbon molecules to produce liquid hydrocarbon fuel.

5. The process of claim 1, further comprising removing from the hydrocarbon slurry (a) the aqueous solution comprising enzymes produced by the microorganisms and (b) the sludge comprising microorganisms and non-hydrocarbon byproducts of enzymatic degradation of cellulose, lignin, xylan and hemicellulose.

6. The process of claim 5, further comprising introducing the removed sludge comprising microorganisms into a second stage bioreactor to cultivate the microorganisms.

7. The process of claim 6, further comprising removing the cultivated microorganisms from the second stage bioreactor and re-introducing the cultivated microorganisms into the first stage bioreactor.

8. The process of claim 5, further comprising removing any remaining hydrocarbon from the aqueous solution comprising enzymes produced by the microorganisms.

9. The process of claim 1, further comprising removing from the first stage bioreactor byproducts selected from the group consisting of oxygen, water and carbon dioxide.

10. The process of claim 1, wherein the microorganisms are selected from the group consisting of mixed ruminal microorganisms, a cellulotyic microorganism, a hemicellulose-degrading bacterial species from an insect gut, a cellulose-degrading fungus from the gut of an insect, a cellulotyic fungus, a hemicellulotyic fungus, a xylan-degrading microorganism, a bacteria from the gut of a wood eating insect, a bacteria from the gut of Loricardiid catfish Panaque, a fungus from the gut of Loricardiid catfish Panaque and a lignan-degrading bacteria isolated from the soil.

11. The process of claim 10, a ruminal microorganism from the mixed ruminal microorganisms is an anaerobic fungi, an anaerobic protozoa, an anaerobic bacteria or an anaerobic Archaebacteria (Archaea).

12. The process of claim 11, wherein the anaerobic Archaebacteria (Archaea) is a strain of Methanobrevibacter ruminatum.

13. The process of claim 10, wherein a ruminal microorganism from the mixed ruminal microorganisms is Alicyclobacillus acidocaldarius, Eubacteria, Ruminococcus albus, R. flavefaciens, Butyrivibrio fibrisolvens, Fibrobacter succinogenes or Selenomonas ruminantium.

14. The process of claim 1, wherein the biomass is agricultural waste or forestry waste.

15. The process of claim 1, wherein the biomass is waste wood from a construction site or a demolition site.

16. The process of claim 1, wherein the biomass is municipal waste.

17. The process of claim 1, wherein the biomass is pulp mill waste or pulp wood waste.

18. The process of claim 1, wherein the biomass is farming debris or yard waste.

19. The process of claim 1, wherein the microorganisms are genetically engineered.

20. The process of claim 19, wherein a strain genetically engineered microorganism produces one length of hydrocarbon molecule.

21. The process of claim 19, wherein the genetically engineered microorganisms are selected from the group consisting of mixed ruminal microorganisms, a cellulotyic microorganism, a hemicellulose-degrading bacterial species from an insect gut, a cellulose-degrading fungus from the gut of an insect, a cellulotyic fungus, a hemicellulotyic fungus, a xylan-degrading microorganism, a bacteria from the gut of a wood eating insect, a bacteria from the gut of Loricardiid catfish Panaque, a fungus from the gut of Loricardiid catfish Panaque and a lignan-degrading bacteria isolated from the soil.

22. The process of claim 21, wherein a ruminal microorganism from the mixed ruminal microorganisms is an anaerobic fungi, an anaerobic protozoa, an anaerobic bacteria or an anaerobic Archaebacteria (Archaea).

23. The process of claim 22, wherein the anaerobic Archaebacteria (Archaea) is a strain of Methanobrevibacter ruminatum.

24. The process of claim 22, wherein the ruminal microorganism from the mixed ruminal microorganisms is Alicyclobacillus acidocaldarius, Ruminococcus albus, R. flavefaciens, Butyrivibrio fibrisolvens, Fibrobacter succinogenes or Selenomonas ruminantium.

25. The process of claim 21, wherein the cellulolytic microorganism is a strain selected from the group consisting of a strain of Eubacteria, a strain of Clostridium, a strain of Ruminococcus, a strain of Caldocellulosiruptor, a strain of Bacteroides, a strain of Acetivibrio, a strain of Thermoactinomyces, a strain of Caldibacillus, a strain of Bacillus, a strain of Acidothermus, a strain of Cellulomonas, a strain of Curtobacterium, a strain of Micromonospora, a strain of Actinoplanes, a strain of Streptomyces, a strain of Thermobifida, a strain of Thermonospora, a strain of Microbispora, a strain of the family Streptosporangiaceae, a strain of Fibrobacter, a strain of Sporocytophaga, a strain of Cytophaga, a strain of Flavobacterium, a strain of Achromobacter, a strain of Xanthomonas, a strain of Cellvibrio, a strain of Pseudomonas and a strain of Myxobacter.

26. The process of claim 20, wherein the hydrocarbon molecule comprises from one to twenty two carbon atoms.

27. The process of claim 25, wherein the hydrocarbon molecule comprises from four to twelve carbon atoms.

28. The process of claim 25, wherein the hydrocarbon molecule comprises eight carbon atoms.

29. The process of claim 1, further comprising manufacturing a polymer, a plastic, a chemical or a solvent from the hydrocarbon molecule.
